(19) United States
(12) Patent Application Publication
(10) Pub. No.: US 2014/0199771 A1
(43) Pub. Date: Jul. 17, 2014

(54) METHOD AND APPARATUS FOR ANALYSIS AND SELECTIVE CATALYTIC REDUCTION OF NOx-CONTAINING GAS STREAMS

(71) Applicant: MIRATECH HOLDINGS, LLC, (US)

(72) Inventors: DANIEL REYNALD PICHE, BROKEN ARROW, OK (US); ADAM J. MERILLAT, TULSA, OK (US)

(73) Assignee: MIRATECH HOLDINGS, LLC, TULSA, OK (US)

(21) Appl. No.: 13/743,789

(22) Filed: Jan. 17, 2013

(51) Int. Cl. G01N 33/00

(52) U.S. Cl. CPC G01N 33/0004
USPC 436/55; 422/83; 436/116

(57) ABSTRACT

An apparatus and method for measuring and controlling the NOₓ and ammonia slip content of a NOₓ-containing gas stream such as, for example, a combustion engine exhaust stream discharged from a Selective Catalytic Reduction (SCR) system. The apparatus includes an analyzer container which preferably has an ammonia slip catalyst element and a pair of automotive type NOₓ sensors positioned therein. One of the NOₓ sensors is positioned before and the other is positioned after the ammonia slip catalyst. The apparatus is heated by positioning the container in the gas stream and can draw a representative gas sample from any size conduit.

### FIELD OF THE INVENTION

The present invention relates to methods and apparatuses for the selective catalytic reduction of nitrogen oxide pollutants (NOₓ) present in combustion engine exhaust streams, other combustion gas streams, chemical process flue or vent gas streams, recycling or recycle gas streams, or any other gas stream which may contain NOₓ. The invention also relates to apparatuses and methods for measuring NOₓ and ammonia slip concentrations in any NOₓ-containing stream of this nature.

### BACKGROUND OF THE INVENTION

Selective catalytic reduction (SCR) is used to reduce nitrogen oxide pollutant (NOₓ) emissions in the exhaust gas streams produced by large combustion engines and in other NOₓ-containing gas streams. Examples of large combustion engines include stationary industrial engines of all types and large diesel engines used in ships, locomotives and other transport vehicles.

By way of example, in an SCR process for a combustion engine exhaust, an ammonia source material is added to the engine exhaust and the exhaust stream is then delivered through an SCR catalyst. Examples of ammonia source materials typically used in SCR processes are urea, anhydrous ammonia, and aqueous ammonia. When the NOₓ components are reacted with urea on the surface of the SCR catalyst, the resulting reaction products are N₂, water, and CO₂. If reacted with anhydrous or aqueous ammonia on the SCR catalyst, the NOₓ emissions are converted to N₂ and water.

It is typically the case in SCR systems that an amount of unreacted urea, ammonia, or other unreacted ammonia source material will pass through the SCR catalyst. This unreacted material which passes through the SCR catalyst is referred to as “ammonia slip.” Ammonia slip can occur, for example, when (a) an excessive amount of the ammonia source material is injected into the exhaust gas, (b) the temperature in the SCR system is too low, and/or (c) the activity of the SCR catalyst has declined due to fouling, degradation or other causes. If released to the atmosphere, the ammonia slip material is toxic and can also react with other atmospheric constituents to form harmful pollutants.

Currently, the amount of urea, ammonia, or other ammonia source material injected into the engine exhaust upstream of the SCR catalyst is controlled based upon either (a) NOₓ concentration measurements taken from the exhaust stream or (b) preexisting performance curves obtained, for example, from the engine manufacturer. Performance curves typically provide predetermined ammonia injection pump rates based upon the engine load.

Some SCR systems are also equipped with an ammonia slip catalyst element located downstream of the SCR catalyst for removing unreacted ammonia in the exhaust effluent. In theory, the addition of the ammonia slip catalyst will allow a greater amount of ammonia to be injected in order to further reduce NOₓ emissions without producing increased ammonia slip emissions. However, current systems which rely upon the over-injection of urea or ammonia to achieve low NOₓ emissions followed by delivering the entire exhaust stream through an ammonia slip catalyst element are disadvantageous because these systems require higher urea consumption, require the addition of an expensive ammonia slip catalyst layer, require a longer SCR reactor structure, and will not control NOₓ emissions at low single digit ppm set point.

A need therefore exists for an improved SCR system which will better control the injection of urea, ammonia, or other ammonia source material into an exhaust stream or other NOₓ-containing gas stream such that (a) low NOₓ emissions can be maintained or further reduced, (b) significant over-injection of the ammonia source material is prevented, and (c) the amount of ammonia slip released to the atmosphere is minimized without having to deliver the exhaust stream or other gas stream through an ammonia slip catalyst element.

In conjunction with these needed improvements, a need also exists for an improved, low cost analyzer which is effective for measuring both NOₓ and ammonia slip levels in an exhaust stream or other NOₓ-containing gas stream with sufficient accuracy to provide enhanced control of the ammonia injection rate while also controlling NOₓ emissions at low set point values.

The analyzers currently available in the art for obtaining reliable NOₓ readings for industrial engines are chemiluminescence units and chemical cell units. Unfortunately these analyzers are expensive and require complicated and/or frequent calibration procedures to provide and maintain an acceptable degree of accuracy. Moreover, neither of these sensors measures the amount of ammonia slip remaining in the exhaust gas downstream of the SCR catalyst.

In contrast to the chemiluminescence and chemical cell analyzers used in some industrial applications, the engine control module NOₓ sensors used in the catalytic converter systems of smaller automobile engines are much less expensive and do not require calibration. Unfortunately, however, these automotive NOₓ sensors have not been suitable for use in large industrial applications.

Automotive-type NOₓ sensors cannot distinguish between NOₓ and ammonia slip present in the gas stream. Rather, the automotive-type sensors simply quantify both the NOₓ and the ammonia slip material present in the gas stream as NOₓ.

In addition, automotive-type NOₓ sensors cannot accurately measure the overall NOₓ concentration of an entire gas stream if the stream is flowing through a conduit which is more than three inches in diameter. In contrast, in an industrial SCR system, the exhaust plenum downstream of the SCR catalyst will commonly have either a circular cross-sectional diameter in the range from about 20 to about 60 inches, or will have a rectangular cross-section wherein the height and the width of the rectangular cross-section are each in the range of from about 24 to about 96 inches.

### SUMMARY OF THE INVENTION

The present invention provides an apparatus and a method which address the needs and alleviate the problems discussed above. The inventive apparatus and method can be used for (a) the selective catalytic reduction of generally any type of combustion engine exhaust or other NOₓ-containing stream, (b) determining both the NOₓ and ammonia slip content of the gas stream, and/or (c) controlling or correcting the injection of ammonia source material in the SCR system. Moreover, in comparison to prior systems, the inventive apparatus and method (i) provide substantially the same degree of accuracy as a chemiluminescence analyzer for measuring NOₓ concentration, (ii) are much less expensive, (iii) also provide ammonia slip concentration and O₂ percent concentration readings, (iv) do not require calibration, (v) do not require sample heating or filtration, and (vi) can be used in an exhaust gas conduit of any size.

Consequently, by way of example, the inventive apparatus and method are well suited for use with industrial or other large combustion engines such as (a) internal or external reciprocating engines which burn diesel, gas, biofuels, and other fossil fuels and (b) combustion turbines which burn gas, oil, biofuels, or other fossil fuels.

In addition, by way of further example, the inventive apparatus and method are well suited for use with NOₓ-containing gases generated by: boilers, burners, flares, cement kilns, other kilns, incinerators, landfill methane recovery, bio reactors, foundries, steel mills, heat treatment furnaces for steel, refineries, nitric acid plants, plants for producing explosives, semiconductor plants, glass smelting, other smelting processes, etc.

In one aspect, there is provided an apparatus for determining a NOₓ content and/or an ammonia slip content of a gas stream. The apparatus preferably comprises: a container; an ammonia slip catalyst positioned in the container; a sample inlet provided in the container upstream of the ammonia slip catalyst for receiving a sample of a gas stream; a first NOₓ sensor having a sensor element positioned in the container upstream of the ammonia slip catalyst; and a second NOₓ sensor having a sensor element positioned in the container downstream of the ammonia slip catalyst. The first and second NOₓ sensors are preferably each of a type which will quantify both NOₓ and ammonia present in the exhaust gas sample as NOₓ. Most preferably, the first and second NOₓ sensors are automotive engine control module NOₓ sensors.

In another aspect, there is provided an apparatus for selective catalytic reduction comprising: a SCR catalyst; a flow passageway extending downstream from the SCR catalyst; a container, at least a portion of the container being positioned in the flow passageway; an ammonia slip catalyst positioned in the container; a sample inlet which is provided in the container upstream of the ammonia slip catalyst and is in fluid communication with the flow passageway for receiving a gas sample from the gas flow passageway; a first NOₓ sensor having a sensor element positioned in the container upstream of the ammonia slip catalyst; and a second NOₓ sensor having a sensor element positioned in the container downstream of the ammonia slip catalyst. The first and second NOₓ sensors are each preferably of a type which will quantify both NOₓ and any ammonia present in the gas sample as NOₓ. Most preferably, the first and second NOₓ sensors are automotive engine control module NOₓ sensors.

In another aspect, there is provided a method of determining a NOₓ content, an ammonia slip content, or both a NOₓ content and an ammonia slip content of a gas stream. The method preferably comprises the steps of: (a) receiving a sample of the gas stream in an analyzer container having an ammonia slip catalyst therein; (b) measuring a first NOₓ content value of the sample in the analyzer container upstream of the ammonia slip catalyst using a first NOₓ sensor which quantifies both NOₓ and any ammonia present in the sample as NOₓ; (c) conducting the sample through the ammonia slip catalyst; and (d) measuring a second NOₓ content value of the sample in the analyzer container downstream of the ammonia slip catalyst using a second NOₓ sensor which quantifies both NOₓ and any ammonia present in the sample as NOₓ.

In another aspect, there is provided a method for the selective catalytic reduction of a gas stream. The method preferably comprises the steps of: (a) adding an ammonia source material to the gas stream at an addition rate; (b) delivering the gas stream through an SCR catalyst; (c) obtaining a sample of the gas stream after step (b); (d) measuring a first NOₓ content value of the sample using a first NOₓ sensor which quantifies both NOₓ and any ammonia present in the sample as NOₓ; (e) delivering the sample through an ammonia slip catalyst after step (d); (f) measuring a second NOₓ content value of the sample after step (e) using a second NOₓ sensor which quantifies both NOₓ and any ammonia present in the sample as NOₓ; and (g) controlling or correcting the addition rate of the ammonia source material used in step (a) based at least in part on the first and the second NOₓ content values measured in steps (d) and (f).

Further aspect features and advantages of the present invention will be apparent to those of ordinary skill in the art upon examining the accompanying drawings and upon reading the following detailed description of the preferred embodiments.

### BRIEF DESCRIPTION OF THE DRAWINGS

FIGS. 1A and 1B schematically illustrate an embodiment 2 of the SCR apparatus and method provided by the present invention.

FIG. 2 schematically illustrates an embodiment 15 of the NOₓ and ammonia slip analyzer provided by the present invention.

### DETAILED DESCRIPTION OF THE PREFERRED EMBODIMENTS

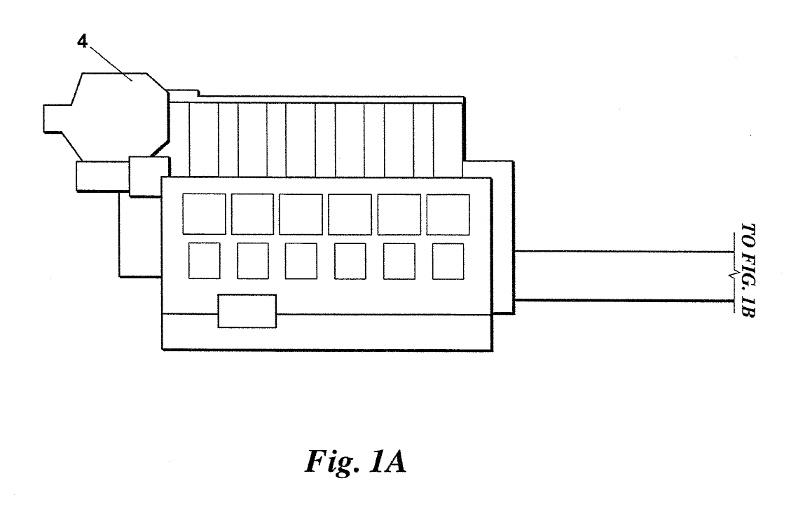

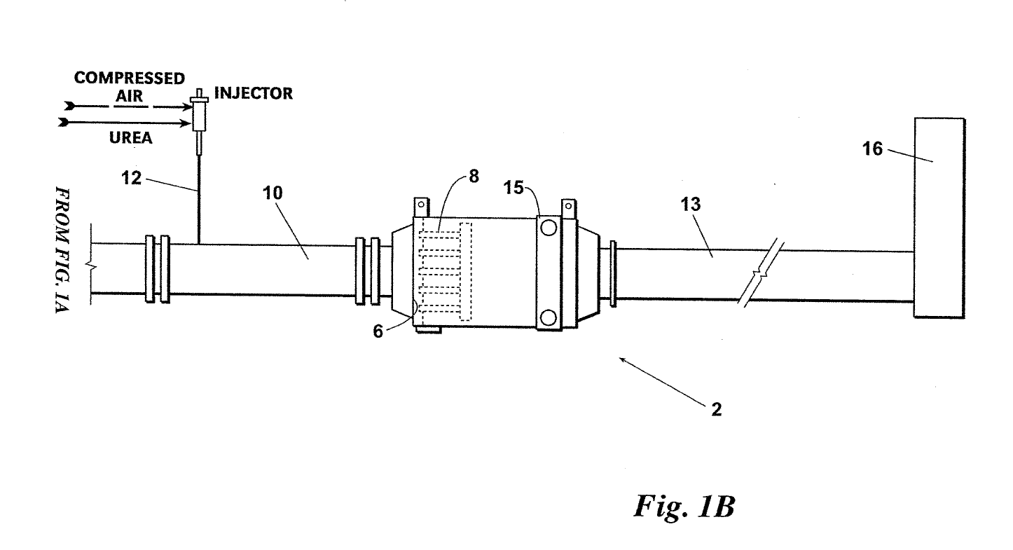

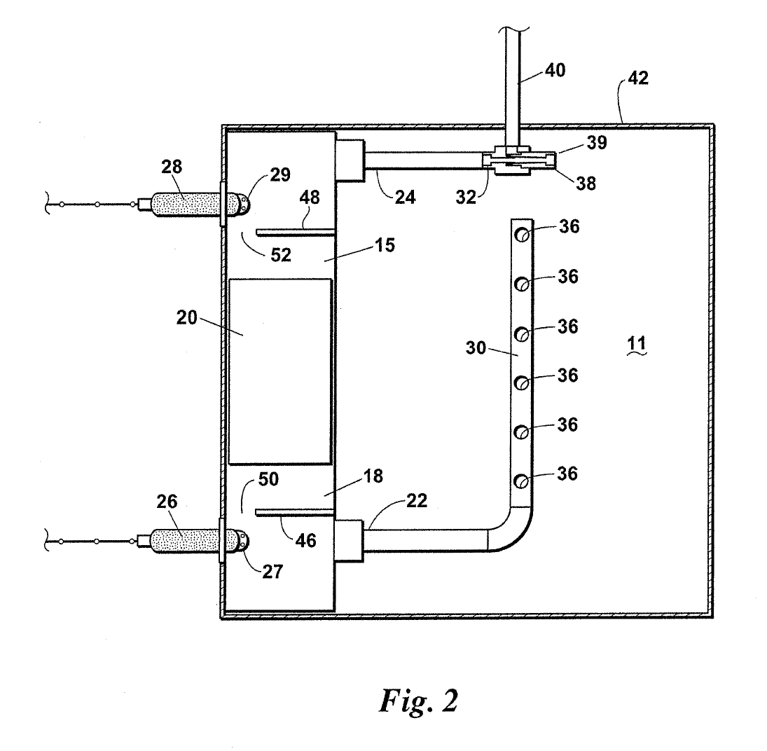

An embodiment 2 of the inventive selective catalytic reduction (SCR) apparatus provided by the present invention for treating a combustion engine exhaust stream is illustrated in FIGS. 1 and 2. However, it will be understood that, as explained above, the applications and uses of the inventive apparatuses and methods described and claimed herein extend well beyond the analysis and treatment of just engine exhaust streams but can be used for any NOₓ-containing gas stream.

The inventive SCR apparatus 2 comprises: an SCR housing 6 having an SCR catalyst element 8 positioned therein; a pipe, housing, duct, manifold, or other conduit 10 which delivers an engine exhaust gas stream from a diesel or other combustion engine 4 to the SCR catalyst housing 6; and injection line 12 for injecting urea, anhydrous ammonia, aqueous ammonia, or other suitable ammonia source material into the conduit 10 upstream of the SCR catalyst element 8; an exhaust gas discharge passageway 11 formed inside the latter portion of the SCR housing 6 and/or inside a discharge conduit 13 connected to the SCR housing 6; an embodiment 15 of an inventive NOₓ and ammonia slip analyzer positioned in the exhaust discharge passageway 11 downstream of the SCR catalyst element 8; and an exhaust stack 16 for discharging the exhaust gas stream to the atmosphere.

As noted above, the engine 4 can be generally any type of industrial, stationary, and/or other large combustion engine. Due to the volume of the exhaust gas which these engines produce, the cross-sectional area of the exhaust discharge passageway 11 wherein the inventive NOₓ and ammonia slip analyzer 15 is located will typically be at least 4 ft² and will more typically be in the range of from 16 ft² to about 100 ft². Consequently, if the exhaust discharge passageway 11 in which the inventive analyzer 15 is located is substantially cylindrical, the cross-sectional diameter of the passageway 11 will typically be at least 20 inches and will more typically be in the range from 36 to about 96 inches. On the other hand, if the exhaust discharge passageway 11 has a substantially rectangular cross-sectional shape, the height and width of the passageway 11 will each typically be at least 20 inches and will more typically be in the range from 36 to about 120 inches.

The SCR catalyst 8 used in the inventive SCR apparatus 2 can be generally any type, structure, and composition of SCR catalyst used in the art for reducing NOₓ emissions in a combustion engine exhaust. Examples include, but are not limited to, vanadium pentoxide, zeolite, zeolite copper, and three-way (TWC) SCR catalysts. SCR catalysts are also commonly supported on substrates such as, for example, ceramic honeycomb and metallic foil materials.

As noted above, the inventive NOₓ and ammonia slip analyzer 15 is installed in the exhaust discharge passageway 11 of the SCR apparatus 2 downstream of the SCR catalyst element 8. As illustrated in FIG. 2, the inventive NOₓ and ammonia slip analyzer 15 preferably comprises: a sealed box or other container 18 which is entirely or at least partially positioned within the exhaust discharge passageway 11 such that the container 18 is contacted and heated by the exhaust gas stream; an ammonia slip catalyst 20 positioned inside the container 18; a container inlet 22 upstream of the ammonia slip catalyst 20 for receiving an exhaust gas sample from the exhaust discharge passageway 11; a container outlet 24 positioned downstream of the ammonia slip catalyst 20 for discharging the sample from the container 18; a first NOₓ sensor 26 having a sensor element 27 positioned in the container 18 upstream of the ammonia slip catalyst 20; a second NOₓ sensor 28 having a sensor element 29 positioned in the container 18 downstream of the ammonia slip catalyst element 20; a sample tube 30 connected to the container inlet 22 for collecting a representative cross-sectional sample of the exhaust gas stream flowing through the exhaust discharge passageway 11; and a suction device 32 connected to the container outlet 24 for drawing the exhaust gas sample into the sample tube 30 and pulling the sample through the analyzer container 18. The suction device 32 preferably discharges the exhaust gas sample back into the exhaust gas stream flowing through the exhaust discharge passageway 11.

The first and second NOₓ sensors 26 and 28 used in the inventive analyzer 15 can be low cost sensors of the type which quantify both NOₓ and unreacted ammonia as NOₓ. Thus, for example, if the exhaust gas sample received in the container inlet 22 has an actual NOₓ concentration of 10 ppm and an actual ammonia concentration of 5 ppm, the reading provided by the first NOₓ sensor 26 will indicate that the NOₓ concentration of the incoming sample is about 15 ppm.

The first and second NOₓ sensors 26 and 28 used in the inventive analyzer 15 will most preferably be automotive engine control module NOₓ sensors. Automotive engine control module NOₓ sensors suitable for use in the inventive NOₓ and ammonia slip analyzer 15 are commercially available, for example, from ECM, Continental, and NGK. The NOₓ sensors 26 and 28 will preferably be electronically linked together by a CAN open bus.

In addition to measuring the NOₓ concentration of the exhaust gas, automotive-type NOₓ sensors 26 and 28 will also measure the actual oxygen concentration of the exhaust gas sample. As will be discussed below, the measurement of the actual oxygen concentration of the exhaust gas can be used along with the NOₓ and ammonia slip readings provided by the inventive analyzer 15 to further enhance the ability of the inventive SCR system to more precisely control the injection of the ammonia source material upstream of the SCR catalyst element 8.

The sample tube 30 used in the inventive analyzer 15 can be generally any type of tube which is effective for drawing a representative sample of the exhaust gas stream across at least most of the cross-section of the exhaust discharge passageway 11. The sample tube 34 preferably includes a plurality of sample inlet openings 36 and will preferably be oriented such that the series of openings 36 extends either vertically, horizontally, or diagonally across at least most of the cross-section of the exhaust discharge passageway 11. The inlet openings 36 will preferably be drilled or other openings which are from about ⅛ to about ½ inch in diameter or width. In addition, the number of inlet openings 36 provided in the series of openings 36 will preferably be in the range from about 2 to about 6 openings 36 per ft.

As noted above, automotive-type NOₓ sensors are not highly effective for direct use in conduits which are more than 3 inches in diameter or width. Consequently, as one alternative, the container 18 of the inventive analyzer 15 can be an elongate container which extends either horizontally or vertically within the exhaust discharge passageway 11 and has either a substantially circular cross-section of not more than 3 inches in diameter or a substantially rectangular cross-section of equivalent size. More preferably, however, the container will have a larger rectangular or similar cross-sectional shape in the range of from about 3×3 inches to about 12×12 inches, most preferably about 6×6 inches, but will also include interior baffles 46 and 48 which increase the sample flow velocity in the vicinity of the NOₓ sensor elements 27 and 29. The interior baffles 46 and 48 preferably form reduced flow areas 50 and 52 in the vicinity of the NOₓ sensor elements 27 and 29 which are each less than 10 in² in size. The baffles 46 and 48 will more preferably form reduced flow areas 50 and 52 which are each less than 9 in² and which are most preferably about 7.5 in² in size.

The analyzer container 18 is preferably formed of stainless steel or other suitable material which is corrosion resistant and will conduct heat energy. The container 18 is entirely or at least partially located inside the exhaust discharge passageway 11 so that the exhaust gas flowing through the exhaust discharge passageway 11 will contact the analyzer container 18 and will heat the container 18 and the ammonia slip catalyst element 20 contained therein. Preferably, a sufficient portion of the container will be contacted and heated by the exhaust gas such that most, and more preferably all or substantially all, of the ammonia slip material in the exhaust gas sample will be reacted as it passes through the ammonia slip catalyst 20. Typically, therefore, the ammonia slip catalyst will be heated to a temperature of at least 500° F. and will preferably to heated to a temperature in the range of from about 500° F. to about 1100° F.

The ammonia slip catalyst 20 used in the inventive NOₓ and ammonia slip analyzer 15 can be of generally any structure and composition effective for removing at least most and preferably all or substantially all of the ammonia slip material present in the exhaust gas sample. Ammonia slip catalysts are readily available in the market and substantially any commercially available ammonia slip catalyst can be used in the inventive NOₓ and ammonia slip analyzer 15. By way of example, but not by way of limitation, ammonia slip catalyst elements are commercially available from Johnson-Matthey, HVG, ECOCAT, and other manufacturers.

The suction device 32 used with the inventive NOₓ ammonia slip analyzer 15 will preferably be a device capable of pulling a sample in the range from about 2 to about 6 actual cubic feet per minute, more preferably about 4 actual cubic feet per minute, through the sample tube 30, the container 18, and the ammonia slip catalyst 20. The suction device 32 will also preferably return the sample to the exhaust gas flow stream.

As illustrated in FIG. 2, the suction device 32 preferably comprises a venturi vacuum element 38 wherein the venturi element 38 and the discharge outlet 39 thereof are positioned within the exhaust discharge passageway 11 downstream of the SCR catalyst element 8. A motive fluid line 40 extends through the wall 42 of the exhaust gas passageway 11 to the venturi vacuum element 38 to deliver a motive fluid, preferably compressed (i.e., pressurized) air, to the venturi device 38 to create a vacuum at the container outlet 24. The amount of compressed air required to operate the venturi vacuum device 38 will typically be in the range from about 2 to about 6 standard cubic feet per minute, more preferably about 4 standard cubic feet per minute, and will simply be discharged along with the used exhaust sample into the exhaust gas flow stream.

In the method of the present invention, the exhaust from the combustion engine 4 flows through the tailpipe or other conduit 10 to the SCR housing 6. To reduce and control NOₓ emissions from the combustion engine 4, urea, anhydrous ammonia, aqueous ammonia, or other ammonia source material is injected into the engine exhaust stream via the injection line 12 and mixes with the exhaust stream as the exhaust stream flows through the conduit 10.

Following the injection of the ammonia source material, the exhaust gas and ammonia source material mixture flow through the SCR catalyst element 8. If the ammonia source material is urea, at least a portion of the NOₓ and urea present in the engine exhaust are converted by contact with the SCR catalyst to nitrogen, water and carbon dioxide. If the ammonia source material is ammonia, at least a portion of the NOₓ and ammonia present in the engine exhaust will be converted to nitrogen and water.

Subsequently, as the treated exhaust gas stream leaves the SCR catalyst element 8 and flows through exhaust discharge passageway 11, the suction element 38 of the inventive NOₓ and ammonia slip analyzer 15 pulls a representative cross-sectional sample of the exhaust gas stream through the sample tube 34 and into the inlet 22 of the analyzer container 18. Inside the inlet portion of the analyzer container 18, the first NOₓ sensor 26 operates to provide an actual NOₓ concentration reading and an actual oxygen concentration reading for the exhaust gas sample. However, because the first NOₓ sensor 26 is not capable of distinguishing NOₓ from ammonia slip material remaining in the exhaust gas, any ammonia slip material in the exhaust gas is also included in the total NOₓ concentration reading provided by the first sensor 26.

Next, the exhaust gas sample is pulled by the suction device 38 through the ammonia slip catalyst element 20. At the same time, the ammonia slip catalyst element 20 is heated by the exhaust gas stream flowing through the exhaust discharge passageway 11 due to the contact between the exhaust gas stream and the analyzer container 18. Thus, all or substantially all of the ammonia slip material present in the exhaust gas sample reacts in the presence of the catalyst 20 and is effectively eliminated from the sample stream.

Consequently, as the sample stream flows out of the ammonia slip catalyst element 20 toward the container outlet 24, the second NOₓ sensor 28 is able to obtain an actual NOₓ concentration reading which cannot be affected by the presence of any significant amount of ammonia slip material. The NOₓ reading provided by the second NOₓ sensor 28 can therefore be subtracted from or otherwise compared to the NOₓ reading provided by the first NOₓ sensor 26 to provide values which are substantially equal to, or which at least sufficiently approximate, the actual NOₓ concentration and ammonia slip concentration of the exhaust gas stream.

In one embodiment of the inventive method, NOₓ and/or ammonia slip concentration values provided by the inventive analyzer 15 can be used in a feedback control loop to directly control the ammonia source injection rate (e.g., the injection pump speed) for the inventive SCR apparatus 2. For example, the corrected NOₓ value provided by subtraction, or by otherwise comparing the NOₓ reading of the second sensor 28 to the NOₓ reading of the first sensor 26, can (by means, e.g., of a proportional-integral-derivative (PID) controller) be used to control the ammonia source injection rate against a NOₓ set point entered by the operator.

Moreover, if the NOₓ control set point for the SCR system is based upon a referenced oxygen percent value for the exhaust which is different from the actual oxygen concentration of the exhaust gas as measured by the inventive analyzer 15, any NOₓ concentration value determined using either or both of the dual NOₓ sensors 26 and 28 of the inventive analyzer 15 can be converted from an actual to a referenced oxygen concentration basis using the following formula:

NOₓ reference=NOₓ actual*(21−reference percent oxygen)/(21−actual percent oxygen).

Alternatively or in addition, the ammonia slip concentration value provided by the inventive analyzer 15 can be used as a feedback correction or control factor, for example, to either (a) correct the ammonia source injection rate against an ammonia slip set point entered by the operator or (b) modify either a predictive or feedback NOₓ control signal for controlling the ammonia source injection rate.

In a presently preferred embodiment of the inventive method, the ammonia source injection rate will be controlled using both (a) a predicted injection rate provided by an injection map based upon the operating load (e.g. KW or brake horsepower) or other output of the engine 4 and (b) one or more feedback correction factors based upon the NOₓ and/or ammonia slip content values provided by the inventive analyzer 15. The predictive injection map for the engine 4 and SCR system 2 will either be provided by the engine manufacturer or will be generated when the engine 4 is installed and commissioned. The map will preferably provide predicted ammonia injection rates for meeting the designated NOₓ set point for the SCR system over a range of engine loads,

By way of example, but not by way of limitation, the feedback correction factor(s) provided in this embodiment by the inventive analyzer 15 could be based upon: (i) a corrected NOₓ content value (with actual O₂ content correction if needed) obtained by comparing the NOₓ reading of the second NOₓ sensor 28 to the NOₓ reading of the first NOₓ sensor 26; (ii) the NOₓ reading provided by the first NOₓ sensor 26 corrected (e.g., either by inclusion in the same control algorithm or separately) by the ammonia slip content value provided by the inventive analyzer 15; and/or (iii) an injection rate correction provided by comparing the ammonia slip content value provided by the inventive analyzer 15 to an ammonia slip set point.

Thus, the present invention is well adapted to carry out the objects and attain the ends and advantages mentioned above as well as those inherent therein. While presently preferred embodiments have been described for purposes of this disclosure, numerous changes and modifications will be apparent to those of ordinary skill in the art. Such changes and modifications are encompassed within the invention as defined by the claims.

What is claimed is:

1. An apparatus for determining a NOₓ content, an ammonia slip content, or both a NOₓ content and an ammonia slip content of a gas stream comprising:
a container;
an ammonia slip catalyst positioned in said container;
said container having a sample inlet upstream of said ammonia slip catalyst for receiving a sample of a gas stream;
a first NOₓ sensor having a sensor element positioned in said container upstream of said ammonia slip catalyst; and
a second NOₓ sensor having a sensor element positioned in said container downstream of said ammonia slip catalyst;
wherein said first and said second NOₓ sensors are each of a type which will quantify both NOₓ and any ammonia present in said sample as NOₓ.

2. The apparatus of claim 1 wherein said first and said second NOₓ sensors are automotive-type NOₓ sensors.

3. The apparatus of claim 1 wherein;
said container further comprises a sample outlet downstream of said ammonia slip catalyst and
said apparatus further comprises a suction device connected to said sample outlet for drawing said sample into said sample inlet, through said ammonia slip catalyst, and out of said sample outlet.

4. The apparatus of claim 3 wherein said suction device comprises a venturi vacuum element.

5. The apparatus of claim 3 further comprising a sampling tube for delivering said sample to said sample inlet of said container, said sampling tube having a series of sample receiving openings provided therein.

6. The apparatus of claim 5 wherein:
said sample receiving openings each have a diameter of at least ¼ inch,
said series of said sample receiving openings extends from a first to a last of said sample receiving openings, and
said series of said sample receiving openings has from about 2 to about 6 of said sample receiving openings per foot.

7. The apparatus of claim 1 wherein said first NOₓ sensor also measures an oxygen concentration value for said sample.

8. An apparatus for selective catalytic reduction comprising:
a SCR catalyst;
a flow passageway extending downstream from said SCR catalyst;
a container, at least a portion of said container being positioned in said flow passageway;
an ammonia slip catalyst positioned in said container;
said container having a sample inlet upstream of said ammonia slip catalyst, said sample inlet being in fluid communication with said flow passageway for receiving a gas sample from said flow passageway;
a first NOₓ sensor having a sensor element positioned in said container upstream of said ammonia slip catalyst; and
a second NOₓ sensor having a sensor element positioned in said container downstream of said ammonia slip catalyst,
wherein said first and said second NOₓ sensors are each of a type which will quantify both NOₓ and any ammonia present in said gas sample as NOₓ.

9. The apparatus of claim 8 wherein said first and said second NOₓ sensors are automotive-type NOₓ sensors.

10. The apparatus of claim 8 wherein:
said container further comprises a sample outlet downstream of said ammonia slip catalyst and said apparatus further comprises a suction device connected to said sample outlet for drawing said gas sample from said gas flow passageway into said sample inlet, through said ammonia slip catalyst, and out of said sample outlet.

11. The apparatus of claim 10 wherein said suction device has a discharge outlet located in said flow passageway.

12. The apparatus of claim 11 wherein said suction device comprises a venturi vacuum element positioned in said flow passageway.

13. The apparatus of claim 12 further comprising a pressurized air line extending into said flow passageway to said venturi vacuum element.

14. The apparatus of claim 10 further comprising a sampling tube for delivering said sample to said sample inlet of said container, said sampling tube having a series of sample receiving openings provided therein and said series of sample receiving openings being positioned in said flow passageway.

15. The apparatus of claim 14 wherein said series of sample receiving openings of said sampling tube extends traversely across at least most of a cross-sectional width, a cross-sectional height, a cross-sectional diagonal dimension, or a cross-sectional diameter of said flow passageway.

16. A method of determining a NOₓ content, an ammonia slip content, or both a NOₓ content and an ammonia slip content of a gas stream comprising the steps of:
(a) receiving a sample of said gas stream in an analyzer container having an ammonia slip catalyst therein;
(b) measuring a first NOₓ content value of said sample in said analyzer container upstream of said ammonia slip catalyst using a first NOₓ sensor which quantifies both NOₓ and any ammonia present in said sample as NOₓ;
(c) conducting said sample through said ammonia slip catalyst; and
(d) measuring a second NOₓ content value of said sample in said analyzer container downstream of said ammonia slip catalyst using a second NOₓ sensor which quantifies both NOₓ and any ammonia present in said sample as NOₓ.

17. The method of claim 16 wherein said ammonia slip catalyst operates in step (c) to convert at least most, if any, ammonia slip material present in said sample to reaction products comprising nitrogen and water.

18. The method of claim 16 wherein said first and said second NOₓ sensors are automotive-type NOₓ sensors.

19. The method of claim 16 further comprising the step of heating said ammonia slip catalyst by contacting at least a portion of said analyzer container with said gas stream.

20. The method of claim 16 wherein:
said analyzer container has a sample outlet downstream of said ammonia slip catalyst and
said sample is drawn into said analyzer container in step (a) and through said ammonia slip catalyst in step (c) using a venturi vacuum element which also pulls said sample out of said sample outlet.

21. The method of claim 20 wherein said venturi vacuum element discharges said sample back into said gas stream.

22. The method of claim 16 further comprising the step of determining a NOₓ content value, an ammonia slip content value, or both for said sample of said gas stream by comparing said second NOₓ content value of said sample to said first NOₓ content value of said sample.

23. The method of claim 16 wherein:
said gas stream is flowing through a flow passageway having a cross-sectional width, a cross-sectional height, a cross-sectional diameter, or other cross-sectional dimension of at least 20 inches and
said sample is drawn into said analyzer container from said flow passageway in step (a) through a sample tube having a series of inlet openings which extend transversely in said flow passageway across at least most of said cross-sectional dimension.

24. A method for selective catalytic reduction of a gas stream comprising the steps of:
(a) adding an ammonia source material to said gas stream at an addition rate;
(b) delivering said gas stream through an SCR catalyst;
(c) obtaining a sample of said gas stream after step (b);
(d) measuring a first NOₓ content value of said sample using a first NOₓ sensor which quantifies both NOₓ and any ammonia present in said sample as NOₓ;
(e) delivering said sample through an ammonia slip catalyst after step (d);
(f) measuring a second NOₓ content value of said sample after step (e) using a second NOₓ sensor which quantifies both NOₓ and any ammonia present in said sample as NOₓ; and
(g) controlling or correcting said addition rate of said ammonia source material used in step (a) based at least in part on a comparison of said first and said second NOₓ content values measured in steps (d) and (f).

25. The method of claim 24 wherein said ammonia slip catalyst operates in step (e) to convert at least most, if any, ammonia slip material present in said sample to other reaction products, said other reaction products comprising nitrogen and water.

26. The method of claim 24 wherein said ammonia source material is urea or ammonia.

27. The method of claim 24 where said first and said second NOₓ sensors are automotive-type NOₓ sensors.

28. The method of claim 24 wherein;
said ammonia slip catalyst is located in an analyzer container;
said first NOₓ sensor has a sensor element located in said analyzer container between said ammonia slip catalyst and an inlet of said analyzer container; and
said second NOₓ sensor has a sensor element located in said analyzer container between said ammonia slip catalyst and an outlet of said analyzer container.

29. The method of claim 28 further comprising the step of heating said ammonia slip catalyst by contacting at least a portion of said analyzer container with said gas stream.

30. The method of claim 28 wherein said sample is obtained in step (c) by drawing said sample into said inlet of said analyzer container using a suction device connected to said outlet of said analyzer container.

31. The method of claim 30 wherein said suction device comprises a venturi vacuum element which returns said sample to said gas stream.

32. The method of claim 28 wherein:
in step (c), said gas stream is flowing through a flow passageway having a cross-sectional width, a cross-sectional height, a cross-sectional diameter, and other cross-sectional dimension of at least 20 inches and
said sample is obtained in step (c) by drawing said sample into said inlet of said analyzer container through a sample tube having a series of inlet openings which extend transversely in said flow passageway across at least most of said cross-sectional dimension.

33. The method of claim 32 wherein at least a portion of said analyzer container is positioned in said flow passageway.
